(19) United States
(12) Patent Application Publication
(10) Pub. No.: US 2024/0238077 A1
(43) Pub. Date: Jul. 18, 2024

(54) NIPPLE RECONSTRUCTION IMPLANT

(71) Applicants: Tepha, Inc., Lexington, MA (US); Cornell University, New York, NY (US)

(72) Inventors: Skander Limem, Lynnfield, MA (US); Kemal Sariibrahimoglu, Sunnyvale, CA (US); Jeffrey Robert Scott, Foxborough, MA (US); Simon F. Williams, Cambridge, MA (US); Jason Spector, New York, NY (US)

(73) Assignees: Tepha, Inc., Lexington, MA (US); Cornell University, New York, NY (US)

(21) Appl. No.: 18/559,853

(22) PCT Filed: May 9, 2022
(86) PCT No.: PCT/US2022/028313

§ 371 Date: Nov. 9, 2023

Related U.S. Application Data
(60) Provisional application No. 63/187,010, filed on May 11, 2021.

(51) Int. Cl. A61F 2/12; A61F 2/00

(52) U.S. Cl. CPC A61F 2/12; A61F 2002/0081; A61F 2210/0004; A61F 2210/0076; A61F 2230/0006; A61F 2230/0069; A61F 2230/0093; A61F 2240/001

(57) ABSTRACT

Absorbable 3D printed implants can be used to reconstruct the nipple with regenerated tissue resulting in improved aesthetic satisfaction. The implants are formed from parallel planes of filaments that are offset and bonded to each other to form macroporous networks (130) with open cell structures comprising cylindrical shapes. The macroporous network (130) may be enclosed by a shell (120) or coating, or may be at least partly filled with a hydrogel. The implants are particularly suitable for use in plastic surgery procedures, for example, to reconstruct the nipple following total mastectomy and breast reconstruction.

### RELATED APPLICATIONS

Foreign priority benefits are claimed under 35 U.S.C. § 119(a)-(d) or 35 U.S.C. § 365(b) of U.S. application No. 63/187,010, filed May 11, 2021.

### FIELD OF THE INVENTION

The present invention generally relates to surgical implants, and more particularly, to 3D printed porous implants suitable for reconstruction of a nipple.

### BACKGROUND OF THE INVENTION

Nipple reconstruction following certain mastectomy procedures has become an important component of breast cancer treatment for some patients because the surgery can provide the patient aesthetic and psychosocial benefits.

Several options for re-creating the appearance of the nipple on the breast are available. These options include prosthetic nipples, made for example from a silicone-based material, that may be temporarily secured to the patient's skin. These prostheses are however external devices attached with a temporary adhesive that will wear out over time, and are perceived as artificial.

Alternatively, a nipple may be reconstructed surgically using the patient's own tissue, or it may be reconstructed using an implant.

Surgically created nipples are permanent and have a more natural feel, but typically require donor skin and a second surgery to harvest suitable tissue. They also require the surgeon to construct a replacement nipple with the proper size, projection and shape which can be challenging when it needs to match the contralateral nipple.

Several nipple reconstruction implants have been developed to avoid the need to harvest suitable tissue for nipple reconstruction from a patient.

US20210052774 to Edwards discloses nipple reconstruction implants derived from acellular tissue matrices and three-dimensional biologic scaffolds.

WO2020081806 to Spector discloses surgical implants for nipple reconstruction comprising minced or zested cartilage that is encaged by an external biocompatible scaffold.

WO2020230997 to Choi discloses an implant for reconstruction of the nipple areolar complex (NAC) comprising a two-wheeled composite with a columnar body and a main body portion.

US2013/0211519 to Dempsey discloses a remodelable implant comprising a remodelable extracellular matrix material, such as an extracellular matrix sheet isolated in sheet form from a mammalian or other tissue source, and configured by rolling and/or molding, to provide a shaped implant.

US2016/0243286 to Collins discloses tissue engineered constructs for nipple reconstruction comprising cells, scaffolding and optionally other materials, such as nutrients and growth factors.

Notwithstanding the above, there is still a need for improved nipple reconstruction implants that, when implanted, can generate new tissue with a specific and desirable appearance and feel.

### SUMMARY OF THE INVENTION

Nipple implants described herein assist the surgeon in reconstructing the nipple-areola complex (NAC) following total mastectomy and breast reconstruction, enhancing the appearance of the breast, reconstructing lost or missing tissue, enhancing the tissue structure of NAC, restoring the natural feeling of soft tissue of the NAC, and delivering biological and synthetic materials to assist in tissue regeneration, repair, and reconstruction of the NAC.

In embodiments, the nipple implants are porous, providing a macroporous network for tissue ingrowth, and may further comprise collagen, cells, and fat. Following implantation, the implant is designed to be invaded by connective tissue, and become well integrated. In embodiments, the nipple implant comprises a cylindrical shape with first and second circular bases of the same circumference at each end of the cylindrical shape.

In embodiments, the implant further comprises a hemispherical shape, or dome shape, connected to the second circular base of the cylindrical shape of the implant.

In embodiments, the implant comprises a shell at least partly surrounding a macroporous network, and the shell comprises a cylindrical shape with first and second circular bases at each end of the cylindrical shape, and a hemispherical shape connected to the second circular base of the cylindrical shape.

In embodiments, the implant has a longitudinal axis with a height h measured longitudinally between a first end of the implant at one end of the axis and a second end of the implant at the opposite end of the axis.

In embodiments, the shell is porous.

In embodiments, the shell does not enclose the macroporous network at the first end of the implant.

In embodiments, the implant further comprises a flange. The flange is located at the first end of the implant. The flange has a larger circumference than the cylindrical shape of the implant so that the flange protrudes from the circular base of the cylindrical shape. In embodiments, the flange is porous. In embodiments, the flange is absorbable. The flange is designed to be placed on the breast mound and posterior to the second end of the implant when the implant with a flange is implanted in a patient.

In embodiments, the implant comprises a cylindrical shape, with first and second circular bases at each end of the cylindrical shape, a hemispherical shape connected to the second circular base of the cylindrical shape, a macroporous network, and optionally a flange located at the first circular base.

In embodiments, the nipple implants comprise a load bearing macroporous network with an open pore structure formed from at least two adjacent parallel planes of filaments bonded to each other. The filaments in each layer extend in the same direction, and are generally parallel to one another. The macroporous network is preferably 3D printed.

In embodiments, the macroporous network of the implant is shaped to fill the shell or cylindrical shape of the implant. In embodiments, the macroporous network has a cylindrical shape connected to a hemispherical shape at one end.

In embodiments, the macroporous network of the implant comprises a first parallel plane of filaments organized in a first geometrical orientation, and a second parallel plane of filaments arranged in a second geometrical orientation to form a porous network with crisscrossed filaments. In embodiments, the macroporous network of the implant further comprises one or more additional parallel planes of filaments arranged in geometrical orientations different from the first and second geometrical orientations. In embodiments, the angles between the filaments in the different parallel planes is between 0 and 135 degrees, preferably, 0, 18, 20, 30, 36, 45 or 60 degrees and more preferably 0, 30, 60, 120 and 0, 45, 90, 135 degrees. In embodiments, the filaments in successive planes are oriented to provide the macroporous network with polygonal pore shapes, including triangular and quadrangular shaped pores. In embodiments, the parallel planes of filaments have the same orientation in adjacent or nonadjacent planes within the macroporous network.

In embodiments, the macroporous network is formed with each subsequent parallel plane of filaments offset from the previous plane of filaments by 18 degrees such that the tenth layer of filaments has the same orientation as the first layer of filaments. In embodiments, the macroporous network is formed with each subsequent parallel plane of filaments offset from the previous plane of filaments by 20 degrees such that the ninth layer of filaments has the same orientation as the first layer of filaments. In embodiments, the macroporous network is formed with each subsequent parallel plane of filaments offset from the previous plane of filaments by 30 degrees such that the sixth layer of filaments has the same orientation as the first layer of filaments. In embodiments, the macroporous network is formed with each subsequent parallel plane of filaments offset from the previous plane of filaments by 36 degrees such that the fifth layer of filaments has the same orientation as the first layer of filaments. In embodiments, the macroporous network is formed with each subsequent parallel plane of filaments offset from the previous plane of filaments by 45 degrees such that the fourth layer of filaments has the same orientation as the first layer of filaments. In embodiments, the macroporous network is formed with each subsequent parallel plane of filaments offset from the previous plane of filaments by 60 degrees such that the third layer of filaments has the same orientation as the first layer of filaments. In the latter case, the angles between the filaments in the different planes are 0, 60 and 120 degrees, and the filaments are oriented in the macroporous network to form pores with a triangular shape.

In embodiments, the filaments of the implant are arranged as chords with endpoints that lie on a circumference of a cylindrical shape of the macroporous network. In embodiments, the filaments of the implant's macroporous network are not continuous. In embodiments, the endpoints of the filaments in a plane of filaments are not connected to another filament that is in the same plane of filaments. In embodiments, the filaments have endpoints on a circumference of the cylindrical shape of the macroporous network, and do not form arcs on a circumference of the cylindrical shape.

In embodiments, the pores of the macroporous network have an average diameter or average width of 75 microns to 10 mm, and more preferably 100 microns to 2 mm, and even more preferably 100 microns to 300 microns.

In embodiments, the filaments of the implant have one or more of the following properties: an average diameter or average width of 10 microns to 5 mm, a breaking load of 0.1 to 200 N, an elongation to break of 10 to 1,000%, and an elastic modulus of 0.05 to 1,000 MPa.

In embodiments, filaments of the implants are formed with surface roughness (Ra). Surface roughness promotes cell attachment and tissue formation on the implants. Surface roughness also promotes attachment of the implant to neighboring tissues, encourages tissue ingrowth, and helps to prevent movement of the device after implantation. In embodiments, the implant comprises filaments having a surface roughness of 0.02 to 75 microns, more preferably 0.1 to 50 or 0.5 to 30 microns, and even more preferably 5 to 30 microns. In embodiments, filaments of the implant are 3D printed with these surface roughness values.

In embodiments, the implant has a shape and size suitable for use in nipple reconstruction. In embodiments, the height h of the implant is 0.1 to 2 cm, more preferably 0.5 to 1.5 cm, and even more preferably 0.3 to 1 cm. In embodiments, the diameter of the cylindrical shape of the implant is from 2 to 10 mm, and more preferably 4 to 7 mm.

In embodiments, the macroporous network of the implant has an infill density of filaments between 1% and 70%, or between 5% and 25%.

In embodiments, the implant comprises a shell or coating that encloses the macroporous network. In embodiments, the shell comprises a stack of concentric filaments.

In embodiments, the shell may be 3D printed with an infill density ranging from 20% to 100%, and more preferably from 50% to 100%. In embodiments, the infill density of the implant may be used to control the rate of absorption of the implant. In embodiments, a high infill shell density may be used to produce an implant with a slower rate of absorption, and low infill shell density may be used to produce an implant with a higher rate of absorption. In embodiments, the shell or coating comprises a foam, an open cell foam, a collagen coating, or a coating comprising poly-4-hydroxybutyrate or copolymer thereof or poly(butylene succinate) or copolymer thereof.

In embodiments, the macroporous network comprises an absorbable polymer. In embodiments, the planes of filaments present in the implant are formed from an absorbable polymer. In embodiments, the absorbable polymer has one or more of the following properties: (i) an elongation at break greater than 100%; (ii) an elongation at break greater than 200%; (iii) a melting temperature of 60° C. or higher; (iv) a melting temperature higher than 100° C.; (v) a glass transition temperature of less than 0° C.; (vi) a glass transition temperature between −55° C. and 0° C.; (vii) a tensile modulus less than 300 MPa; and (viii) a tensile strength higher than 25 MPa. In embodiments, the absorbable polymer comprises, or is prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 4-hydroxybutyric acid, 4-hydroxybutyrate, 3-hydroxyoctanoate, c-caprolactone, 1,4-butanediol, 1,3-propane diol, ethylene glycol, glutaric acid, malic acid, malonic acid, oxalic acid, succinic aid, and adipic acid, or the absorbable polymer comprises poly-4-hydroxybutyrate (P4HB) or copolymer thereof, or poly(butylene succinate) (PBS) or copolymer thereof. In embodiments, the implant comprises P4HB and copolymers thereof, or PBS and copolymers thereof, and is not crosslinked. In embodiments, the PBS polymer and copolymers may further comprise one or more of the following: branching agent, cross-linking agent, chain extender agent, and reactive blending agent. The PBS and P4HB polymers and copolymers may be isotopically enriched. In embodiments, the polymers used to prepare the implants have weight average molecular weights of 50 to 1,000 kDa, more preferably 90 to 600 kDa, and even more preferably from 200 to 450 kDa relative to polystyrene determined by GPC.

In embodiments, the implant is absorbable. The implants preferably comprise a polymeric material with a predictable rate of degradation, and a predictable strength retention in vivo. When the implants are absorbable, degradation of the implant can allow further invasion of the implant with tissue, and this process can continue until the implant is completely absorbed.

In embodiments, the implant further comprises one or more of the following: autologous fat, fat lipoaspirate, injectable fat, adipose cells, fibroblast cells, stem cells, gels, hydrogels, hyaluronic acid, collagen, antimicrobial agent, antibiotic agent, and bioactive agent.

In embodiments, the implants have anisotropic properties meaning that the implants have different properties in different directions.

In embodiments, the implant is shell-less, and optionally the perimeter edges of the implant are treated, for example, to remove barbs and make the implant generally smoother. The edges may be treated by, for example, trimming or heat treating.

In embodiments, the implant retains strength long enough to allow new tissue to fill the space occupied by the implant, and thereby maintain the shape of the nipple after implantation of the implant. The implant directs re-modeling of the patient's tissue to form the nipple. The implant preferably provides support for the nipple during this transition period. The shape of the nipple implant is maintained for a prolonged period in order to direct tissue ingrowth into the implant, and produce the desired nipple shape.

In embodiments, the macroporous network of the implant is at least partly filled with a degradable polymer. The degradable polymer is preferably degraded faster than the macroporous network. In embodiments, the macroporous network comprises a hydrogel.

In embodiments, the implants have an endotoxin content of less than 20 endotoxin units per implant.

In embodiments, the implants are sterilized implants. The implants can be sterilized by a range of techniques including without limitation ethylene oxide, electron beam, or gamma-irradiation.

In embodiments, the implant is formed using a process selected from the following group: forming the macroporous network by 3D printing the parallel planes of filaments; forming the macroporous network by melt extrusion deposition printing; and forming the macroporous network by bonding the filaments in adjacent parallel planes by 3D printing.

In embodiments, methods are provided for manufacturing a nipple implant comprising a macroporous network with an open cell structure, optionally at least partly surrounded by a shell, wherein the implant comprises a cylindrical shape with first and second circular bases with the same circumferences, a hemispherical shape connected to the second circular base, a flange located at the first end of the implant and extending beyond the cylindrical shape of the implant, and a longitudinal axis with a first end and a second end, wherein the macroporous network comprises at least two adjacent parallel planes of filaments bonded to each other, and wherein the method comprises forming the macroporous network by one of the following: forming at least two parallel planes of filaments from a polymeric composition by 3D printing of the filaments; and forming at least two parallel planes of filaments from a polymeric composition by melt extrusion deposition printing. In embodiments, the methods provide for forming the implant with a compressive modulus of 0.1 kPa to 10 MPa at 5 to 15% strain, more preferably 5 to 500 kPa at 5 to 15% strain, and even more preferably 10 to 200 kPa at 5 to 15% strain.

In embodiments, the method of manufacturing the nipple implant comprises at least partly enclosing the macroporous network in a shell by 3D printing filament in concentric circles to enclose the macroporous network. In embodiments, the method of manufacturing the nipple implant further comprises enclosing the macroporous network at least partly in a shell by coating the macroporous network with a polymeric composition.

In embodiments, the methods of manufacturing the nipple implant comprise 3D printing a porous flange protruding from the circular base of the first end of the implant's cylindrical shape.

In embodiments, the methods of manufacturing the nipple implant comprise 3D printing a porous shell to at least partly surround the macroporous network.

In embodiments, the method of manufacturing comprises 3D printing the filaments of the macroporous network in a cylindrical shape as chords with endpoints that lie on the circumference of the cylindrical shape. In embodiments, the method of manufacturing comprises printing the filaments as chords so that within a plane of filaments the filaments are not connected to each other by a filament in the same plane as the plane of filaments. In embodiments, the method of manufacturing the implant comprises 3D printing the filaments with endpoints on the circumference of the cylindrical shape of the macroporous network without forming arcs with the filaments on the circumference of the cylindrical shape.

In embodiments, the method of manufacturing further comprises at least partly filling the macroporous network with a hydrogel by printing a hydrogel in the macroporous network, injecting a hydrogel into the macroporous network, or coating a hydrogel onto the macroporous network.

In embodiments, the method of manufacturing the implant comprises printing at least two parallel planes of filaments with an angle between the filaments in the parallel planes selected from one of the following: between 1 and 90 degrees, or 18, 20, 30, 36, 45, 60 or 90 degrees.

In embodiments, the method of manufacturing the implant comprises 3D printing the macroporous network with an infill density of filaments of between 1% and 60%, or between 5% and 25%.

In embodiments, the methods of manufacturing the implants comprise forming the parallel planes of filaments by 3D printing from a polymeric composition selected from a polymer or copolymer comprising, or prepared from, one or more of the following monomers: glycolide, lactide, glycolic acid, lactic acid, 1,4-dioxanone, trimethylene carbonate, 3-hydroxybutyric acid, 3-hydroxybutyrate, 4-hydroxybutyric acid, 4-hydroxybutyrate, c-caprolactone, 1,4-butanediol, 1,3-propane diol, ethylene glycol, glutaric acid, malic acid, malonic acid, oxalic acid, succinic acid, and adipic acid, or wherein the polymeric composition comprises poly-4-hydroxybutyrate or copolymer thereof, or poly(butylene succinate) or copolymer thereof.

In embodiments, the methods of manufacturing the implants comprise forming the filaments of the macroporous network from a polymer with one or more of the following properties: (i) an elongation at break greater than 100%; (ii) an elongation at break greater than 200%; (iii) a melting temperature of 60° C. or higher, (iv) a melting temperature higher than 100° C., (v) a glass transition temperature of less than 0° C., (vi) a glass transition temperature between −55° C. and 0° C., (vii) a tensile modulus less than 300 MPa, and (viii) a tensile strength higher than 25 MPa. In preferred embodiments, the macroporous network of the implant is made from P4HB, PBS, P4HB copolymers or PBS copolymers, by 3D printing. In embodiments, the method of manufacturing the implant comprises forming the filaments of the macroporous network by 3D printing with one or more of the following properties: (i) an elongation at break greater than 100%; (ii) an elongation at break greater than 200%; (iii) a melting temperature of 60° C. or higher, (iv) a melting temperature higher than 100° C., (v) a glass transition temperature of less than 0° C., (vi) a glass transition temperature between −55° C. and 0° C., (vii) a tensile modulus less than 300 MPa, and (viii) a tensile strength higher than 25 MPa.

In embodiments, the methods of manufacturing the implants comprise 3D printing the macroporous network, and adding one or more of the following components: autologous fat, fat lipoaspirate, injectable fat, adipose cells, fibroblast cells, stem cells, gel, hydrogel, hyaluronic acid, collagen, antimicrobial, antibiotic, and bioactive agent. In embodiments, these components are added to the macroporous network by coating, spraying, immersion or injection.

In embodiments, the implant is implanted by a method comprising: making an incision in a patient to create a tissue enclosure that is configured to receive a nipple implant; and inserting the nipple implant into the tissue enclosure, wherein the tissue enclosure is configured to conform around the nipple implant. In embodiments, the method of implanting the implant comprises configuring an incision to create tissue flaps with opposable edges, such that when the edges are brought together the tissue flaps form a void for receiving the nipple implant so that the inner surface of the tissue flaps are in contact with the nipple implant. In embodiments, the method of implanting the implant comprises making an incision with a CV-flap incision path, a S-flap incision path, or a star-flap incision path. In embodiments, the implant comprises a flange protruding from the cylindrical shape of the implant, and the implant is implanted in the patient with the flange positioned on the breast mound of the patient and posterior to the second end of the cylindrical shape. In embodiments, the implant comprises a hemispherical shape, and the implant is implanted so that the hemispherical shape is adjacent to the skin of the patient and anterior to the remainder of the implant.

In embodiments, the implant serves to provide the surgeon with a means to deliver cells, stem cells, differentiated cells, fat cells, muscle cells, platelets, tissues, lipoaspirate, extracellular adipose matrix proteins, gels, hydrogels, hyaluronic acid, collagen, bioactive agents, drugs, antibiotics, and other materials to the implant site.

In embodiments, the implants can be implanted to replace and or increase a soft tissue volume or a tissue mass.

These advantages as well as other objects and advantages of the present invention will become apparent from the detailed description to follow, together with the accompanying drawings.

### BRIEF DESCRIPTION OF THE DRAWINGS

FIG. 1A is a front view a nipple implant 100 with a first end 116 and a second end 117 and a height h measured between the first and second ends, a cylindrical shape 110 with a first circular base 111 and a second circular base 112 with a distance between the circular bases 113, a hemispherical or dome shape 140 with height 141 connected to the second circular base 112, a shell 120 with pores 121 and outer diameter 114 defining the shell circumference, and a flange 150 of outer diameter 151 and thickness 152 connected to the first end of the implant. The implant has a longitudinal axis 115. A macroporous network 130 is partially visible through the pores 121 in the shell.

FIG. 1B is a bottom view of the nipple implant 100 shown in FIG. 1A showing the circumference 153 of the macroporous network 130 within the shell, the outer diameter 154 of the macroporous network, and the outer diameter 151 of the flange. The flange 150 is shown with pores 155.

FIG. 1C is an isometric view of the nipple implant 100 shown in FIG. 1A showing the cylindrical shape 110, pores 121 of the shell, and the flange 150.

FIG. 2A is a front view of implant 100 shown in FIG. 1A in accordance with one embodiment of the invention.

FIG. 2B is a cross sectional view taken along line A-A of a nipple implant 100 shown in FIG. 2A in accordance with one embodiment of the invention. The nipple implant is shown with a shell 120 with shell thickness 210 and shell pores 121, a flange 150, a macroporous network 130 formed with filaments 131 inside the shell.

FIG. 2C is an enlarged view of the macroporous network 130 shown as Detail C in FIG. 2B showing filaments 131 that form the macroporous network.

FIG. 2D is an enlarged view of the shell 120 shown as Detail B in FIG. 2A showing a pore 121 in the shell, and the macroporous network 130 inside the shell.

FIG. 3A is a front view of nipple implant 100 shown in FIG. 1A in accordance with one embodiment of the invention.

FIG. 3B is a cross sectional view taken along line F-F of a nipple implant 100 shown in FIG. 3A showing the shell 120, flange 150, and layers of parallel filaments 131, 132 and 133 arranged with angles between the layers of parallel filaments of 60 degrees to crisscross the stack layers of parallel filaments.

FIG. 3C is a cross sectional view taken along line E-E of a nipple implant 100 shown in FIG. 3A showing the shell 120, flange 150, pore 121 in the shell, and the macroporous network 130 inside the shell formed from layers of parallel filaments arranged at angles of 60 degrees to each other.

FIG. 4 is a picture of a bottom view of a 3D printed nipple implant with an internal filament structure made from printed P4HB filaments, no flange, and a shell with 100% infill.

FIG. 5 is a picture of a top side perspective view of a 3D printed nipple implant shown in FIG. 4 with an internal filament structure made from printed P4HB filaments.

FIGS. 6A-6I are pictures of 3D printed nipple implants, with 20, 25 and 30% infill, and no outer shell.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

To further assist in understanding the following definitions are set forth below. However, it is also to be appreciated that unless defined otherwise as described herein, all technical and scientific terms used herein have the same meaning as commonly understood by one of ordinary skill in the art to which this invention belongs.

### I. Definitions

“Absorbable” as generally used herein means the material is degraded in the body, and the degradation products are eliminated or excreted from the body. The terms “absorbable”, “resorbable”, “degradable”, and “erodible”, with or without the prefix “bio”, can be used interchangeably herein, to describe materials broken down and gradually absorbed, excreted, or eliminated by the body, whether degradation is due mainly to hydrolysis or mediated by metabolic processes.

“Bioactive agent” as generally used herein refers to therapeutic, prophylactic or diagnostic agents, preferably agents that promote healing and the regeneration of host tissue, and also therapeutic agents that prevent, inhibit or eliminate infection. “Agent” includes a single such agent and is also intended to include a plurality.

“Biocompatible” as generally used herein means the biological response to the material or implant being appropriate for the implant's intended application in vivo. Any metabolites of these materials should also be biocompatible.

“Blend” as generally used herein means a physical combination of different polymers, as opposed to a copolymer formed of two or more different monomers.

“Compressive modulus” as used herein is measured with a universal testing machine at a cross-head speed of 20 mm min-. Implants are preloaded to engage the load and compressed at 5 to 15% strain with the load applied along the longitudinal axis of the implant. Clinically relevant cyclic load is repeated 10 times and compressive modulus is calculated based on secondary cyclic load due to the artifact caused by a take up of slack, and alignment or seating of the specimen. Compressive modulus may also be measured using ASTM standards ASTM D1621-16 or ASTM D695-15.

“Copolymers of poly-4-hydroxybutyrate” as generally used herein means any polymer containing 4-hydroxybutyrate with one or more different hydroxy acid units. The copolymers may be isotopically enriched.

“Copolymers of poly(butylene succinate)” as generally used herein means any polymer containing 1,4-butanediol and succinic acid units, and one or more different diol or diacid units, or hydroxy acid units. The copolymers may include one or more of the following: branching agent, cross-linking agent, chain extender agent, and reactive blending agent. The copolymers may be isotopically enriched.

“Endotoxin content” as generally used herein refers to the amount of endotoxin present in an implant or sample, and is determined by the limulus amebocyte lysate (LAL) assay.

“Infill density” as used herein is the ratio of volume occupied by the printed material in a porous implant divided by the total volume occupied by the printed material and the pore space expressed as a percentage.

“Molecular weight” as generally used herein, unless otherwise specified, refers to the weight average molecular weight (Mw), not the number average molecular weight (Mn), and is measured by GPC relative to polystyrene.

“Poly(butylene succinate) mean a polymer containing 1,4-butanediol units and succinic acid units. The polymer may include one or more of the following: branching agent, cross-linking agent, chain extender agent, and reactive blending agent. The polymer may be isotopically enriched.

“Poly(butylene succinate) and copolymers” includes polymers and copolymers prepared with one or more of the following: chain extenders, coupling agents, cross-linking agents and branching agents.

“Poly-4-hydroxybutyrate” as generally used herein means a homopolymer containing 4-hydroxybutyrate units. It can be referred to herein as P4HB or TephaFLEX® biomaterial (manufactured by Tepha, Inc., Lexington, MA). The polymers may be isotopically enriched.

“Soft tissue” as used herein means body tissue that is not hardened or calcified. Soft tissue excludes hard tissues such as bone and tooth enamel.

“Strength retention” refers to the amount of time that a material maintains a particular mechanical property following implantation into a human or animal. For example, if the tensile strength of a resorbable fiber or strut decreases by half over 3 months when implanted into an animal, the fiber or strut's strength retention at 3 months would be 50%.

“Surface roughness” (Ra) as used herein is the arithmetic average of the absolute values of the profile height deviations from a mean line, recorded within an evaluation length.

### II. Materials for Preparing Implants

In embodiments, the implants can be used to form a nipple, reshape a nipple, reconstruct a nipple, modify a nipple, or replace a nipple that has been damaged or surgically removed. The implants can eliminate the need for donor site surgery during nipple reconstruction. The implants are biocompatible, and are preferably replaced in vivo by the patient's tissue as the implants degrade. The implants have a compressive modulus suitable for reconstruction of the nipple. Optionally, the implants can be coated or filled with a hydrogel, bioactive agent, autologous tissue, autologous fat, fat lipoaspirate, injectable fat, adipose cells, fibroblast cells, and stem cells prior to implantation, during implantation, or post-implantation.

### A. Polymers for Preparing Implants

In embodiments, the implants contain macroporous networks that are formed from at least two parallel layers of filaments bonded together. In embodiments, the filaments in a first layer have a first orientation, and the filaments in a second layer have a second orientation that is different to the first orientation. In embodiments, the filaments in the first and second layers of the macroporous network are crisscrossed. In embodiments, the macroporous network may comprise additional layers of filaments with different orientations to the first and second orientations of filaments. In embodiments, the adjacent layers of filaments are bonded to each other at multiple points where they crisscross. In embodiments, pores are formed between the filaments of the macroporous network. The dimensions of the pores are dependent upon the number and direction of the filaments in the macroporous network, the spacing of the filaments, and the size and shape of the filaments. A macroporous network may comprise two or more parallel layers of filaments bonded together, but preferably 20, 30, 40, 50 or more layers of filaments.

The macroporous network of the implant may comprise permanent materials, such as non-degradable thermoplastic polymers, including polymers and copolymers of ethylene and propylene, including ultra-high molecular weight polyethylene, ultra-high molecular weight polypropylene, nylon, polyesters such as poly(ethylene terephthalate), poly(tetrafluoroethylene), polyurethanes, poly(ether-urethanes), poly(methylmethacrylate), polyether ether ketone, polyolefins, and poly(ethylene oxide). However, the macroporous network of the implant preferably comprises absorbable materials, more preferably thermoplastic or polymeric absorbable materials, and even more preferably the implant and the implant's macroporous network are made completely from absorbable materials.

In a preferred embodiment, the implant's macroporous network is made from one or more absorbable polymers or copolymers, preferably absorbable thermoplastic polymers and copolymers, and even more preferably absorbable thermoplastic polyesters. The implant's macroporous network may, for example, be prepared from polymers including, but not limited to, polymers comprising glycolic acid, glycolide, lactic acid, lactide, 1,4-dioxanone, trimethylene carbonate, 3-hydroxybutyric acid, 4-hydroxybutyrate, 3-hydroxyhexanoate, 3-hydroxyoctanoate, E-caprolactone, including polyglycolic acid, polylactic acid, polydioxanone, polycaprolactone, copolymers of glycolic and lactic acids, such as VICRYL® polymer, MAXON® and MONOCRYL® polymers, and including poly(lactide-co-caprolactones); poly(orthoesters); polyanhydrides; poly(phosphazenes); polyhydroxyalkanoates; synthetically or biologically prepared polyesters; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); silk (including recombinant silks and silk derivatives and analogs); chitin; chitosan; modified chitosan; biocompatible polysaccharides; hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide), or polycaprolactone and copolymers thereof, including random copolymers and block copolymers thereof.

Preferably the macroporous network of the implant is prepared from an absorbable polymer or copolymer that will be substantially absorbed after implantation within a 1 to 24-month timeframe, more preferably a 3 to 18-month timeframe, and retain some residual strength for at least 2 weeks to 6 months.

Blends of polymers and copolymers, preferably absorbable polymers, can also be used to prepare the implant's macroporous network. Particularly preferred blends of absorbable polymers are prepared from absorbable polymers including, but not limited to, polymers comprising glycolic acid, glycolide, lactic acid, lactide, 1,4-dioxanone, trimethylene carbonate, 3-hydroxybutyric acid, 4-hydroxybutyrate, C-caprolactone, 1,4-butanediol, 1,3-propane diol, ethylene glycol, glutaric acid, malonic acid, oxalic acid, succinic aid, adipic acid, or copolymers thereof.

In a particularly preferred embodiment, poly-4-hydroxybutyrate (Tepha's P4HB™ polymer, Lexington, MA) or a copolymer thereof is used to make the implant's macroporous network. Copolymers include P4HB with another hydroxy acid, such as 3-hydroxybutyrate, and P4HB with glycolic acid or lactic acid monomer. Poly-4-hydroxybutyrate is a strong, pliable thermoplastic polyester that is biocompatible and resorbable (Williams, et al. Poly-4-hydroxybutyrate (P4HB): a new generation of resorbable medical devices for tissue repair and regeneration, Biomed. Tech. 58(5):439-452 (2013)).

Upon implantation, P4HB hydrolyzes to its monomer, and the monomer is metabolized via the Krebs cycle to carbon dioxide and water. In a preferred embodiment, the P4HB homopolymer and copolymers thereof have a weight average molecular weight, Mw, within the range of 50 kDa to 1,200 kDa (by GPC relative to polystyrene), more preferably from 100 kDa to 600 kDa, and even more preferably 200 kDa to 450 kDa. A weight average molecular weight of the polymer of 50 kDa or higher is preferred for processing and mechanical properties.

In another preferred embodiment, the macroporous network of the implant is prepared from a polymer comprising at least a diol and a diacid. In a particularly preferred embodiment, the polymer used to prepare the macroporous network is poly(butylene succinate) (PBS) wherein the diol is 1,4-butanediol and the diacid is succinic acid. The poly(butylene succinate) polymer may be a copolymer with other diols, other diacids or a combination thereof. For example, the polymer may be a poly(butylene succinate) copolymer that further comprises one or more of the following: 1,3-propanediol, ethylene glycol, 1,5-pentanediol, glutaric acid, adipic acid, terephthalic acid, malonic acid, methylsuccinic acid, dimethylsuccinic acid, and oxalic acid. Examples of preferred copolymers are: poly(butylene succinate-co-adipate), poly(butylene succinate-co-terephthalate), poly(butylene succinate-co-butylene methylsuccinate), poly(butylene succinate-co-butylene dimethylsuccinate), poly(butylene succinate-co-ethylene succinate) and poly(butylene succinate-co-propylene succinate). In embodiments, the polymer may be a poly(butylene succinate) copolymer further comprising a hydroxy acid. Examples of hydroxy acids are: glycolic acid and lactic acid. The poly(butylene succinate) polymer or copolymer may also further comprise one or more of the following: chain extender, coupling agent, cross-linking agent and branching agent. For example, poly(butylene succinate) or copolymer thereof may be branched or cross-linked by adding one or more of the following agents: malic acid, trimethylol propane, glycerol, trimesic acid, citric acid, glycerol propoxylate, and tartaric acid. Particularly preferred agents for branching or crosslinking the poly(butylene succinate) polymer or copolymer thereof are hydroxycarboxylic acid units. Preferably the hydroxycarboxylic acid unit has two carboxylic groups and one hydroxyl group, two hydroxyl groups and one carboxyl group, three carboxyl groups and one hydroxyl group, or two hydroxyl groups and two carboxyl groups. In one preferred embodiment, the implant's macroporous network is prepared from poly(butylene succinate) comprising malic acid as a branching or cross-linking agent. This polymer may be referred to as poly(butylene succinate) cross-linked with malic acid, succinic acid-1,4-butanediol-malic acid copolyester, or poly(1,4-butylene glycol-co-succinic acid), cross-linked with malic acid. It should be understood that references to malic acid and other cross-linking agents, coupling agents, branching agents and chain extenders include polymers prepared with these agents wherein the agent has undergone further reaction during processing. For example, the agent may undergo dehydration during polymerization. Thus, poly(butylene succinate)-malic acid copolymer refers to a copolymer prepared from succinic acid, 1,4-butanediol and malic acid. In an embodiment, the poly(butylene succinate)-malic acid copolymer may further comprise one or more hydroxy acids, such as glycolic acid and lactic acid. In another preferred embodiment, malic acid may be used as a branching or cross-linking agent to prepare a copolymer of poly(butylene succinate) with adipate, which may be referred to as poly[(butylene succinate)-co-adipate]cross-linked with malic acid. As used herein, “poly(butylene succinate) and copolymers” includes polymers and copolymers prepared with one or more of the following: chain extenders, coupling agents, cross-linking agents and branching agents. In a particularly preferred embodiment, the poly(butylene succinate) and copolymers thereof contain at least 70%, more preferably 80%, and even more preferably 90% by weight of succinic acid and 1,4-butanediol units. The polymers comprising diacid and diols, including poly(butylene succinate) and copolymers thereof and others described herein, preferably have a weight average molecular weight (Mw) of 10,000 to 400,000, more preferably 50,000 to 300,000 and even more preferably 100,000 to 200,000 based on gel permeation chromatography (GPC) relative to polystyrene standards. In a particularly preferred embodiment, the polymers and copolymers have a weight average molecular weight of 50,000 to 300,000, and more preferably 75,000 to 300,000. In one preferred embodiment, the poly(butylene succinate) or copolymer thereof used to make the macroporous network has one or more, or all of the following properties: density of 1.23-1.26 g/cm³, glass transition temperature of −31° C. to −35° C., melting point of 113° C. to 117° C., melt flow rate (MFR) at 190° C./2.16 kgf of 2 to 10 g/10 min, and tensile strength of 30 to 60 MPa.

In another embodiment, the polymers and copolymers described herein that are used to prepare the macroporous network of the implant, including P4HB and copolymers thereof and PBS and copolymers thereof, include polymers and copolymers in which known isotopes of hydrogen, carbon and/or oxygen are enriched. Hydrogen has three naturally occurring isotopes, which include ¹H (protium), ²H (deuterium) and ³H (tritium), the most common of which is the ¹H isotope. The isotopic content of the polymer or copolymer can be enriched for example, so that the polymer or copolymer contains a higher than natural ratio of a specific isotope or isotopes. The carbon and oxygen content of the polymer or copolymer can also be enriched to contain higher than natural ratios of isotopes of carbon and oxygen, including, but not limited to ¹³C, ¹⁷O or ¹⁸O. Other isotopes of carbon, hydrogen and oxygen are known to one of ordinary skill in the art. A preferred hydrogen isotope enriched in P4HB or copolymer thereof or PBS or copolymer thereof is deuterium, i.e. deuterated P4HB or copolymer thereof or deuterated PBS or copolymer thereof. The percent deuteration can be up to at least 1% and up to 5, 10, 15, 20, 25, 30, 35, 40, 45, 50, 55, 60, 65, 70, 75, 80 or 85% or greater.

In a preferred embodiment, the polymers and copolymers that are used to prepare the macroporous network, including P4HB and copolymers thereof and PBS and copolymers thereof, have low moisture contents. This is preferable to ensure the implants can be produced with high tensile strength, prolonged strength retention, and good shelf life. In a preferred embodiment, the polymers and copolymers that are used to prepare the implants have a moisture content of less than 1,000 ppm (0.1 wt %), less than 500 ppm (0.05 wt %), less than 300 ppm (0.03 wt %), more preferably less than 100 ppm (0.01 wt %), and even more preferably less than 50 ppm (0.005 wt %).

The compositions used to prepare the implants desirably have a low endotoxin content. In preferred embodiments, the endotoxin content is low enough so that the implants produced from the polymer compositions have an endotoxin content of less than 20 endotoxin units per device as determined by the limulus amebocyte lysate (LAL) assay. In one embodiment, the polymeric compositions used to prepare the macroporous network of the implant have an endotoxin content of <2.5 EU/g of polymer or copolymer. For example, the P4HB polymer or copolymer, or PBS polymer or copolymer have an endotoxin content of <2.5 EU/g of polymer or copolymer.

### B. Additives

Certain additives may be incorporated into the implant, preferably in the polymeric compositions that are used to make the macroporous network. In one embodiment, these additives are incorporated with the polymers or copolymers described herein during a compounding process to produce pellets that can be subsequently processed to produce the macroporous networks. For example, pellets may be extruded or printed to form the filaments of the macroporous networks. In another embodiment, the pellets may be ground to produce powders suitable for further processing, for example, by 3D printing. Or, powders suitable for further processing, for example by 3D printing, may be formed directly by blending the additives and polymer or copolymer. If necessary, powders used for processing may be sieved to select an optimum particle size range. In another embodiment, the additives may be incorporated into the polymeric compositions used to prepare the macroporous networks of the implants using a solution-based process.

In a preferred embodiment, the additives are biocompatible, and even more preferably the additives are both biocompatible and absorbable.

In one embodiment, the additives may be nucleating agents and/or plasticizers. These additives may be added to the polymeric compositions used to prepare the macroporous networks of the implants in sufficient quantity to produce the desired result. In general, these additives may be added in amounts between 1% and 20% by weight. Nucleating agents may be incorporated to increase the rate of crystallization of the polymer, copolymer or blend. Such agents may be used, for example, to facilitate fabrication of the macroporous network, and to improve the mechanical properties of the macroporous network. Preferred nucleating agents include, but are not limited to, salts of organic acids such as calcium citrate, polymers or oligomers of PHA polymers and copolymers, high melting polymers such as PGA, talc, micronized mica, calcium carbonate, ammonium chloride, and aromatic amino acids such as tyrosine and phenylalanine.

Plasticizers that may be incorporated into the polymeric compositions for preparing the macroporous networks of the implants include, but are not limited to, di-n-butyl maleate, methyl laureate, dibutyl fumarate, di(2-ethylhexyl) (dioctyl) maleate, paraffin, dodecanol, olive oil, soybean oil, polytetramethylene glycols, methyl oleate, n-propyl oleate, tetrahydrofurfuryl oleate, epoxidized linseed oil, 2-ethyl hexyl epoxytallate, glycerol triacetate, methyl linoleate, dibutyl fumarate, methyl acetyl ricinoleate, acetyl tri(n-butyl) citrate, acetyl triethyl citrate, tri(n-butyl) citrate, triethyl citrate, bis(2-hydroxyethyl) dimerate, butyl ricinoleate, glyceryl tri-(acetyl ricinoleate), methyl ricinoleate, n-butyl acetyl rincinoleate, propylene glycol ricinoleate, diethyl succinate, diisobutyl adipate, dimethyl azelate, di(n-hexyl) azelate, tri-butyl phosphate, and mixtures thereof. Particularly preferred plasticizers are citrate esters.

### C. Bioactive Agents, Cells and Tissues

The implants can be loaded, filled, coated, or otherwise incorporated with bioactive agents. Bioactive agents may be included in the implants for a variety of reasons. For example, bioactive agents may be included in order to improve tissue in-growth into the implant, to improve tissue maturation, to provide for the delivery of an active agent, to improve wettability of the implant, to prevent infection, and to improve cell attachment. The bioactive agents may also be incorporated into the macroporous network of the implant.

The implants can contain active agents designed to stimulate cell in-growth, including growth factors, cell adhesion factors including cell adhesion polypeptides, cellular differentiating factors, cellular recruiting factors, cell receptors, cell-binding factors, cell signaling molecules, such as cytokines, and molecules to promote cell migration, cell division, cell proliferation and extracellular matrix deposition. Such active agents include fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insulin-like growth factor (IGF), hepatocyte growth factor (HGF), interleukin-1-B (IL-1 B), interleukin-8 (IL-8), and nerve growth factor (NGF), and combinations thereof. As used herein, the term “cell adhesion polypeptides” refers to compounds having at least two amino acids per molecule that are capable of binding cells via cell surface molecules. The cell adhesion polypeptides include any of the proteins of the extracellular matrix which are known to play a role in cell adhesion, including fibronectin, vitronectin, laminin, elastin, fibrinogen, collagen types I, II, and V, as well as synthetic peptides with similar cell adhesion properties. The cell adhesion polypeptides also include peptides derived from any of the aforementioned proteins, including fragments or sequences containing the binding domains.

The implants can incorporate wetting agents designed to improve the wettability of the surfaces of the macroporous networks to allow fluids to be easily adsorbed onto the implant surfaces, and to promote cell attachment and or modify the water contact angle of the implant surface. Examples of wetting agents include polymers of ethylene oxide and propylene oxide, such as polyethylene oxide, polypropylene oxide, or copolymers of these, such as PLURONICS®. Other suitable wetting agents include surfactants, emulsifiers, and proteins such as gelatin.

The implants can contain gels, hydrogels or living hydrogel hybrids to further improve wetting properties and to promote cellular growth throughout the macroporous network structures of the implants. Hydrogel hybrids consist of living cells encapsulated in a biocompatible hydrogel, for example, gelatin, methacrylated gelatin (GelMa), silk gels, and hyaluronic acid (HA) gels.

Other bioactive agents that can be incorporated in the implants include antimicrobial agents, in particular antibiotics, disinfectants, oncological agents, anti-scarring agents, anti-inflammatory agents, anesthetics, small molecule drugs, anti-adhesion agents, inhibitors of cell proliferation, anti-angiogenic factors and pro-angiogenic factors, immunomodulatory agents, and blood clotting agents. The bioactive agents may be proteins such as collagen and antibodies, peptides, polysaccharides such as chitosan, alginate, hyaluronic acid and derivatives thereof, nucleic acid molecules, small molecular weight compounds such as steroids, inorganic materials such as hydroxyapatite and ceramics, or complex mixtures such as platelet rich plasma. Suitable antimicrobial agents include: bacitracin, biguanide, triclosan, gentamicin, minocycline, rifampin, vancomycin, cephalosporins, copper, zinc, silver, and gold. Nucleic acid molecules may include DNA, RNA, siRNA, miRNA, antisense or aptamers.

The implants may also contain allograft material and xenograft materials, including acellular dermal matrix material and small intestinal submucosa (SIS).

In another embodiment, the implants may incorporate systems for the controlled release of the therapeutic or prophylactic agents.

In an embodiment, the implants are coated with autograft, allograft or xenograft tissue and cells prior to implantation, during implantation, or after implantation, or any combination thereof. The autologous tissue and cells are preferably one or more of the following: autologous fat, fat lipoaspirate, fat tissue, injectable fat, adipose tissue, adipose cells, fibroblast cells, and stem cells. As will be evident herein, the macroporous network structures of the implants are designed to create not only the shape of a nipple implant, but also a large surface area that can retain tissue and cells to encourage tissue ingrowth.

### III. Methods for Preparing Implants

A variety of methods can be used to manufacture the implants.

In embodiments, the implant is prepared so that it is able to provide one or more of the following: (i) structural support, (ii) a macroporous network scaffold for tissue ingrowth, (iii) a macroporous network scaffold for delivering cells, tissues, collagen, hyaluronic acid, and bioactive agents, including fat, lipoaspirate, adipose cells, fibroblast cells, and stem cells (iv) a structure that can provide mechanical spacing, (v) a structure that can be coated with cells, tissues, collagen, hyaluronic acid, and bioactive agents, including fat, lipoaspirate, adipose cells, fibroblast cells, and stem cells on the inside of the macroporous network by injection using a needle, and (vi) a structure with a compressive modulus of 0.1 kPa to 10 MPa at 5 to 15% strain, or more preferably 5 to 500 kPa at 5 to 15% strain.

### A. Implant Shapes

In an embodiment, the implants are designed so that when manufactured, they are three-dimensional. In embodiments, the implants are designed to be used for the reconstruction of the nipple of the NAC. In embodiments, the implants are designed to create a nipple with a specific shape, size and projection. In embodiments, the implants are designed to create a nipple that matches the contralateral nipple in terms of shape, projection, size and position.

The implant's shape allows the surgeon to increase tissue volume, reconstruct lost or missing tissue or tissue structures, contour tissues, augment tissues, restore nipple function, repair damaged tissue structures, enhance an existing tissue structure, and alter the projection of the nipple. In a preferred embodiment, the implants are used to reconstruct the nipple following mastectomy. In an embodiment, the implants allow the shape of soft tissue structures to be altered, or sculpted, without the use of permanent implants.

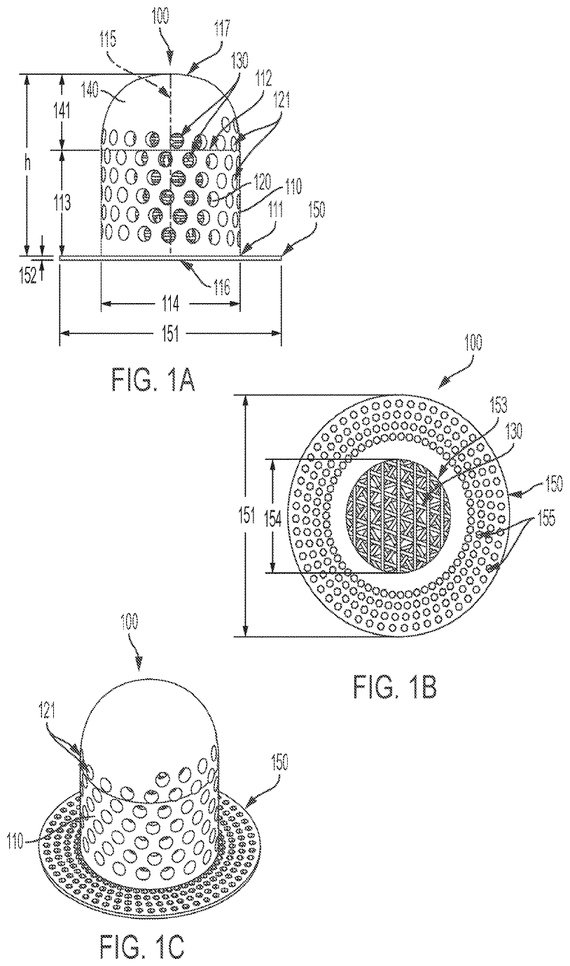

In embodiments, and with reference to FIG. 1A, a nipple implant 100 comprises a first end 116, a second end 117, a height h measured between the first and second ends, a cylindrical shape 110 with a first circular base 111 and a second circular base 112 with a distance between the circular bases 113, a hemispherical or dome shape 140 with height 141 connected to the second circular base 112, a shell 120 with pores 121 and outer diameter 141 defining the shell and implant circumference, and a flange 150 of outer diameter 151 and thickness 152 connected to the first end of the implant. The implant 100 comprises a longitudinal axis 115, and a macroporous network 130. The macroporous network 130 is partially visible in FIG. 1A through the pores 121 in the implant's shell.

FIG. 1B is a bottom view of the implant 100 shown in FIG. 1A. Flange 150 defines an aperture through which the macroporous network 130 is visible inside the implant's shell. The macroporous network 130 has a circumference 153 and a diameter 154. The flange 150 is also shown with pores 155, and an outer diameter 151.

FIG. 1C is an isometric view of the nipple implant 100 shown in FIG. 1A, and shows the implant's cylindrical shape 110, the pores 121 in the implant's shell, and the flange 150.

The cylinder and hemispherical shapes of the nipple implant are shaped to provide diameter and projection to the nipple. By “projection” of the implant it is meant the maximum distance h between the first 116 and second 117 ends of the implant.

In embodiments, the implant has bullet, flange-cylinder, or top-hat type shape. In other embodiments, the implant does not comprise a hemispherical shape at the second end of the cylindrical shape. In embodiments, the implant has a cylindrical shape, or a cylindrical shape with a flange at one end.

In embodiments, the implant does not comprise a flange component protruding from the circular base on the first end of the cylindrical shape. In embodiments, the flange component is porous. In embodiments, the flange is not porous.

In embodiments, the implant is shell-less. In embodiments, the shell completely surrounds the macroporous network. In embodiments, the shell partially surrounds the macroporous network. In embodiments, the shell does not enclose the first end 116 of the implant.

The implant can be assembled or printed to have any size suitable for use as a nipple implant.

In embodiments, the dimensions of the implant may be shaped and sized to create a nipple that matches the contralateral nipple in terms of shape, projection, and size. Preferably, the implant provides symmetry in the size, shape and position of the reconstructed nipple to match a contralateral nipple.

In embodiments, the nipple implants may be sized or shaped to provide a low, moderate or high projection of the nipple. In embodiments, the height h measured between the first end and second end of the implant is 0.1 to 2 cm, more preferably 0.5 to 1.5 cm, and even more preferably 0.3 to 1 cm. The projection of the nipple may also be controlled by selecting the diameter of the cylindrical shape of the implant. In embodiments, the diameter of the first and second bases of the cylindrical shape of the implant is from 2 to 10 mm, and more preferably 4 to 7 mm.

### B. Construction of the Implants

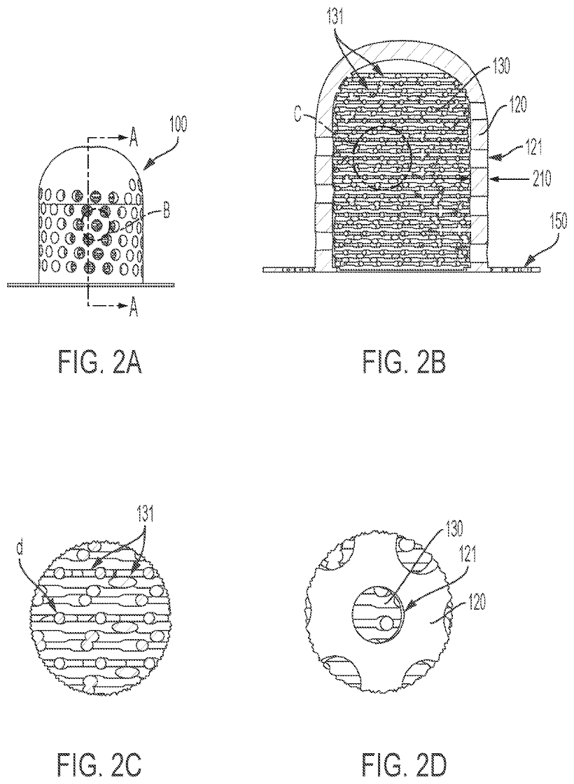

In embodiments, the nipple implants comprise a load bearing macroporous network with an open pore structure. The macroporous network comprises filaments. FIG. 2B is a cross sectional view taken along line A-A of the nipple implant 100 shown in FIG. 2A in accordance with an embodiment of the invention, and shows the macroporous network 130 of the implant inside the implant's shell 120. The macroporous network comprises filaments 131. The shell is shown with a shell thickness 210 and shell pores 121. The implant is shown with a flange 150. An enlarged view of the macroporous network 130 is shown as detail C in FIG. 2C, and shows the filaments 131 of the macroporous network. FIG. 2D shows an enlargement of detail B of FIG. 2A including the shell 120, pore 121 in the shell, and the macroporous network 130 inside the shell.

In embodiments, the macroporous network is formed from at least two adjacent parallel planes of filaments bonded to each other. The filaments in each layer extend in the same direction, and are generally parallel to one another.

In embodiments, the macroporous network is 3D printed.

In embodiments, the filaments of the macroporous network are applied or printed in separate or individual layers (e.g., one layer at a time on top of each other, namely, stacked). A second layer of filaments having filaments oriented in a second direction, or angle, are applied on top of a first layer of filaments, wherein the first layer of filaments is oriented in a first direction or angle. Additional layers of filaments may be added to build up a porous structure comprising layers of filaments. Applying layers of filaments in this manner, having different orientations, creates a crisscross, triangular, square, quadrilateral, parallelogram, or other polygon-like open pore structure when viewed from the top or bottom of the macroporous network of the implant.

The number of layers having different orientation or printer angles (when the implant is 3D printed) may vary. In embodiments, 2-3 different types of layer orientations are applied. However, in other embodiments, 3-5, or more different types of layer orientations or print angles are provided.

Within a single layer of filaments of the macroporous network, each filament can have the same orientation or direction. For example, the filaments in each layer may extend in the same direction and are generally parallel to one another.

In embodiments, the angles between successive layers of parallel filaments may range from 0 to 179 degrees, but is more preferably 0 to 90 degrees, and even more preferably 0 to 60 degrees.

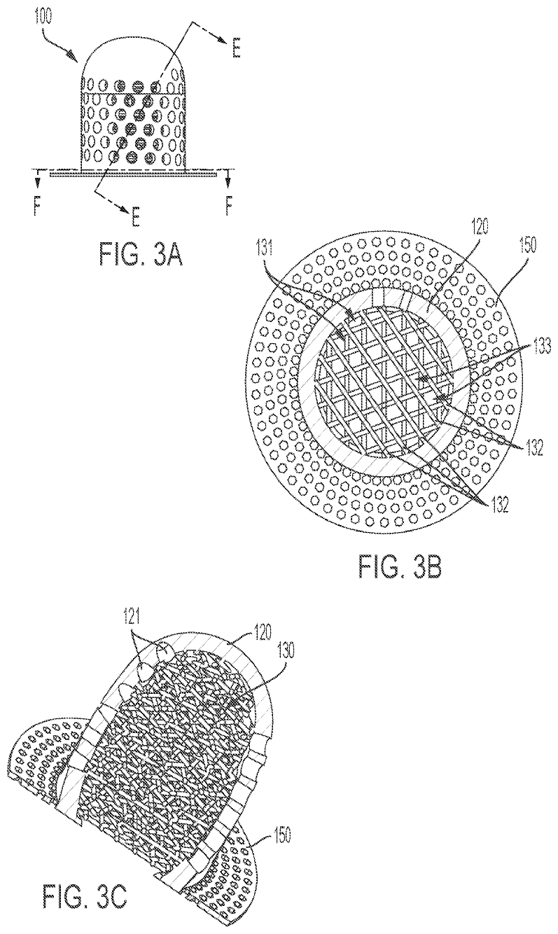

FIG. 3B is a cross sectional view taken along line F-F of nipple implant 100 shown in FIG. 3A showing in one embodiment layers of parallel filaments 131, 132, and 133 arranged with angles between the layers of parallel filaments of 60 degrees. The arrangement of filaments 131, 132 and 133 provides a crisscrossed structure of stacked layers of parallel filaments. FIG. 3B also shows the implant's flange 150 and shell 120. A cross sectional view of the implant taken along line E-E of the nipple implant 100 shown in FIG. 3A is shown in FIG. 3C, and shows the macroporous network 130 inside the shell 120 of the implant. FIG. 3C also shows the flange 150, and pores 121 in the shell of the implant.

In embodiments, the implants are constructed with layers of filaments, and the filaments in the layers are arranged as chords. In this embodiment, the layers are formed, for example, by printing separate filaments as chords rather than by printing a continuous filament to form the filaments in a layer. Thus, in this embodiment, the filaments in a layer are not connected within the layer to each other, and the filaments do not form arcs on the circumference of the cylindrical shape. Instead, the filaments have endpoints on the circumference of the cylindrical shape of the implant's macroporous network. Forming the implant's macroporous network with chords of filament provides a more porous implant structure than forming the macroporous network with filament forming arcs on the circumference of the implant or macroporous network. A more porous network is advantageous in promoting tissue ingrowth into the implant.

In embodiments, implants with different compressive modulus values may be constructed by varying the angles between successive layers of parallel filaments. For example, the angles may be varied to form implants with compressive modulus values ranging from 0.1 kPa to 10 MPa at 5 to 15% strain, more preferably 1 to 10 MPa at 5 to 15% strain, and even more preferably 1 to 5 MPa at 5 to 15% strain.

In embodiments, the implant comprises layers of parallel filaments with at least one layer of parallel filaments angled at 1-60 degrees from another layer of parallel filaments. In embodiments, the implant comprises layers of filaments where the parallel filaments of a first layer are angled at an angle (a) from an adjacent layer of filaments, where α is a multiple of 2, 3 or 5 between 0 and 60 degrees. In embodiments, angle α is 18, 20, 24, 30, 36, 45 or 60 degrees, from another adjacent layer of parallel filaments.

In embodiments, the distances between the filaments in a layer are equal. However, in other embodiments (not shown), the distances between filaments within a single layer are not equal, and may vary within the layer, or vary from layer to layer.

In embodiments, the macroporous network of the implant comprises at least two layers of filaments bonded to each other. In other embodiments, all layers of filaments in the macroporous network are bonded to at least one other layer of filaments.

In embodiments, implant macroporous networks with at least two adjacent parallel planes of filaments bonded to each other may be prepared with the filaments in adjacent or nonadjacent planes having the same orientation as each other, or different orientations to each other. Forming macroporous networks comprising filaments in adjacent layers with the same orientations to each other may be used to increase the porosity of the implant or to alter the compression modulus of the implant.

In embodiments, the three-dimensional architecture of the implant's macroporous network may comprise two or more adjacent layers of parallel filaments where there is no offset or angle between the layers of parallel filaments. In these embodiments, filaments in an adjacent layer of the macroporous network are placed on top of each other so that there is no angle between them, and so that they do not form a crisscross structure.

Incorporating sections of adjacent layers where the filaments in each layer have the same orientation may be used to produce implants with larger pore sizes. For example, an implant may be formed where successive layers of parallel filaments are first angled from the prior layers by 60 degrees, followed by a section where adjacent layers are not angled, followed by successive layers again angled at 60 degrees to the prior layer. The architecture used to prepare the implant's macroporous network may be selected based on the desired properties of the implant. For example, the filaments in each layer may be printed at 0, 60, and 120-degree angles to each other forming a triangular open pore structure as shown in FIG. 3B.

Repeated printing of layers before changing the print angle may also be used to change the compressive modulus of the macroporous network of the implant. For example, two filament layers may be printed at an angle of 0 degrees, the print angle then changed and two filament layers printed at an angle of 60 degrees before two filament layers are printed at another angle such as e.g., an angle of 120 degrees. The process may then be repeated to build up the porous structure to the desired dimensions. In order to create even larger pore sizes, multiple layers (for example, 3, 4, 5, 6, 7, 8, 9, 10 or more) may be printed at the same angle (i.e., repeated) before the print angle is changed. It is to be understood that in accordance with the invention, these angles may be varied to form different shaped open pore structures with two or more filament layers printed at the same angle before the print angle is changed.

In embodiments, the macroporous networks of the implants have pores with widths or diameters of 75 μm to 10 mm, and more preferably 100 μm to 2 mm. In embodiments, the pore sizes of the macroporous network of the implant are the same. In embodiments, the macroporous network of the implant comprises a mixture of pore sizes.

Preferably, the macroporous networks of the implants have an architecture that provides a large surface area and large void volume suitable to allow the macroporous network to be colonized by cells and invaded by tissue.

In embodiments, the average diameters of the filaments are 50 to 800 μm, more preferably 100 to 600 μm, and even more preferably 150 to 550 μm. In embodiments, the distances between the filaments of the implant are between 50 μm and 1 mm, more preferably 100 μm and 1 mm, and even more preferably 200 μm and 1 mm. The average diameters of the filaments and the distances between the filaments may be selected according to the properties of the implant's macroporous network that are desired, including the compression modulus, the porosity, and the infill density, defined as the ratio of volume occupied by filament material in the implant's macroporous network divided by the total volume of the macroporous network expressed as a percentage. In embodiments, the infill density of the implant's macroporous network is from 1 to 60%, and more preferably from 5 to 25%.

In embodiments, the architecture of the implant's macroporous network preferably provides sufficient porosity to makes it possible to coat the inside of the macroporous network with allograft or xenograft cells, preferably autologous cells, including, but not limited to, autologous fat, fat lipoaspirate, lipo-filling, injectable fat, fibroblast cells, and stem cells. The architecture of the implant's macroporous network is also preferably designed to allow the inner surfaces of the macroporous network to be coated with collagen and or hyaluronic acid or derivative thereof.

In embodiments, the dimensions of the pores of the implant's macroporous network are large enough to allow needles to be inserted into the pores of the macroporous network in order to deliver bioactive agents, cells, fat and other compositions by injection. In embodiments, the architecture of the macroporous network is designed to allow needles with gauges of 12-21 to be inserted into the macroporous network. This property allows the macroporous network to be loaded with cells, collagen, bioactive agents and additives, including fat, using a syringe and without damaging the macroporous network. Preferably, the macroporous networks allow insertion of needles into the open pore structure with outer diameters of 0.5 to 3 mm.

The porosity and shapes of the pores of the implant's macroporous network may be tailored by changing the offset or angle between the filaments in each layer.

In embodiments, the implant's shell may be prepared from a stack of concentric filaments at the periphery of the implant's macroporous network enclosing successive layers of parallel filaments.

In embodiments, the macroporous network of the implant comprises an external shell (e.g., shell 120,) or coating. In embodiments, the shell has an outer surface and an inner surface that surrounds an interior volume of said shell. The external shell or coating may partially or fully encase the filaments of the implant's macroporous network. In embodiments, the thickness of the shell or coating is from 10 μm to 5 mm, and more preferably 100 μm to 1 mm. In embodiments, the shell is formed from concentric stacks of a filament at the periphery of the stacked layers of parallel filaments. The shell may be 3D printed. In embodiments, the thickness of the shell comprises 2, 3, 4, 5 or more filaments side by side. In embodiments, the shell is 3D printed, and has an infill density of 20% to 100% or more preferably 50% to 100%. In embodiments, the infill density of the implant may be used to control the rate of absorption of the implant. In embodiments, a high infill shell density may be used to produce an implant with a slower rate of absorption, and low infill shell density may be used to produce an implant with a higher rate of absorption. In embodiments, the macroporous network is coated with a polymeric composition.

In embodiments, the shell or coating is permeable to a needle.

In embodiments, the shell comprises a foam with interconnected pores. In embodiments, the shell is an open cell foam, more preferably an open cell foam comprising poly-4-hydroxybutyrate or copolymer thereof or poly(butylene succinate) or copolymer thereof.

In embodiments, the shell comprises collagen, and more preferably type I collagen. In embodiments, the shell comprises collagen, and is 0.1 to 5 mm, or more preferably 0.5 to 3 mm in thickness.

In embodiments, the implant comprises layers of parallel filaments with at least one layer of parallel filaments angled at 1-60 degrees from another layer of parallel filaments, and wherein the implant further comprises a shell surrounding the layers of parallel filaments. In embodiments, the implant comprises layers of parallel filaments with each layer of parallel filaments angled at 1-60 degrees, more preferably 18, 20, 30, 36, 45 or 60 degrees, from another adjacent layer of parallel filaments, and wherein the implant further comprises a shell surrounding the layers of parallel filaments.

In embodiments, the implant comprises a shell wherein the shell has been heat treated to minimize the roughness of the outer surface of the shell.

In embodiments, the implant comprises a hemispherical shape at the second end of the cylindrical shape that is formed by 3D printing. The hemispherical shape may be formed from filaments. In embodiments, the hemispherical shape and the cylindrical shape enclose the macroporous network, and the macroporous network extends inside the cylindrical shape to inside the hemispherical shape. In embodiments, the print pattern of the macroporous network occupying the interior of the cylindrical shape is the same as the print pattern of the macroporous network occupying the interior of the hemispherical shape.

In embodiments, the implant comprises a flange protruding from the first circular base of the cylindrical shape that is formed by 3D printing. The flange component is preferably formed from a porous network of filaments.

In one embodiment, the implant is prepared using 3D printing to construct the implant's macroporous network. 3D Printing of the macroporous network is highly desirable since it allows precise control of the shape of the implant's macroporous network. Suitable methods for 3D printing include fused filament fabrication, fused pellet deposition, melt extrusion deposition, selective laser melting, printing of slurries and solutions using a coagulation bath, and printing using a binding solution and granules of powder. Preferably, the macroporous network of the implant is prepared by melt extrusion deposition.

The nipple implant depicted in FIGS. 1A-C, 2A-D and 3A-C can be manufactured by melt extrusion deposition. The implant can be printed with different filling densities and with different angles between the filaments. As described above, in embodiments the infill density of the implant's macroporous network is from 1 to 60%, and more preferably from 5 to 25%, the average diameters of the filaments are 50 to 800 μm, more preferably 100 to 600 μm, and even more preferably 150 to 550 μm, the distances between the filaments of the implant are between 50 μm and 1 mm, more preferably 100 μm and 1 mm, and even more preferably 200 μm and 1 mm, and the angles between the filaments in adjacent layers may range from 0 to 179 degrees, but are more preferably 0 to 90 degrees, and even more preferably 0 to 60 degrees. These parameters may be selected according to the properties desired for the macroporous network or implant, including the compression modulus and porosity. For example, the porosity of the macroporous network may be decreased by decreasing the infill density if the filament sizes, spacing between filaments, and print pattern are kept constant. As the infill density decreases, the compression modulus also decreases if the filament sizes, spacing between filaments, and print pattern are kept constant. An exemplary infill range for the body of the implant is 1 to 50, and more preferably 5 to 20%. An exemplary infill range for the shell of the implant is 50% to 100%, and more preferably 80 to 100%.

In a typical procedure, the implant is prepared by melt extrusion deposition of a composition comprising an absorbable polymer or blend thereof.

The absorbable polymer or blend is preferably dried prior to printing to avoid a substantial loss of intrinsic viscosity. Preferably, the polymer or blend is dried so that the moisture content of the composition to be printed is no greater than 0.5 wt. % as measured gravimetrically, and more preferably no greater than 0.05 wt. %. The polymer or blend may be dried in vacuo. In a particularly preferred method, the polymer or blend is dried in a vacuum chamber under a vacuum of at least 10 mbar, more preferably of at least 0.8 mbar, to a moisture content of less than 0.03% by weight. Elevated temperatures below the melting point of the polymer may also be used in the drying process. Alternatively, the polymer may be dried by extraction into a solvent and re-precipitation of the polymer, or with the use of desiccants. The moisture content of the polymer or blend may be determined using a VaporPro Moisture Analyzer from Arizona Instruments, or similar instrument.

In an embodiment, the implant is formed by melt extrusion deposition of poly-4-hydroxybutyrate (P4HB). P4HB polymer (Mw of 100-600 kDa) is pelletized prior to melt extrusion deposition, and preferably dried as described above. A suitable 3D printer for printing the implant's macroporous network is an Arburg Freeformer 3D printer. P4HB pellets may be 3D printed to form the nipple implant (e.g. 100) with the macroporous network (e.g. 130) (as shown in the examples of FIGS. 1-3) using, for example, the printing parameters shown in Table 1 and the Arburg Freeformer 3D printer, and a 3D CAM (Computer Aided Design Model) for the implant. The average diameters of the 3D filaments that are printed from the P4HB polymer are selected based upon the properties of the implant desired, including the implant's compression modulus, and porosity or fill density (i.e. the number of 3D printed filaments per mm between the contours of the 3D printed device). Preferably, the average filament diameters or widths are 50 to 800 μm, more preferably 100 to 600 μm, and even more preferably 150 to 550 μm.

TABLE 1
Parameters for Melt Extrusion Deposition Printing
of P4HB Nipple Implant Macroporous Networks

| | |
|---|---|
| Print head temp (° C.) | 185 |
| Barrel zone 2 (° C.) | 135 |
| Barrel zone 1 (° C.) | 100 |
| Build chamber temp (° C.) | 10-20° C. |
| Screw speed (m/min) | 4 |
| Back pressure (Bar) | 50 |
| Recovery stroke (mm) | 6 |
| Deco speed (mm/s) | 2 |
| Deco stroke (mm) | 4 |
| Discharge nr (%): | 40-80 |
| In Filling density Shell (%) | 30-100 |
| In Filling density Body (%) | 1-50 |
| Drop ratio | 1-1.6 |

In another embodiment, the parameters shown in Table 2 may be used to 3D print the implant using a composition comprising poly(butylene succinate) or copolymer thereof.

TABLE 2
Parameters for Melt Extrusion Deposition Printing
of PBS Nipple Implant Macroporous Networks

| | |
|---|---|
| Print head temp (° C.) | 185-200 |
| Barrel zone 2 (° C.) | 135-150 |
| Barrel zone 1 (° C.) | 110 |
| Build chamber temp (° C.) | 10-50 |
| Screw speed (m/min) | 4 |
| Back pressure (Bar) | 50 |
| Recovery stroke (mm) | 6 |
| Deco speed (mm/s) | 2 |
| Deco stroke (mm) | 4 |
| Discharge nr (%): | 60-75 |
| In Filling density Shell (%) | 30-100 |
| In Filling density Body (%) | 1-50 |
| Drop ratio | 1-1.6 |

### C. Properties of the Implant

In embodiments, the mechanical properties of the macroporous network and optional shell are designed to provide an implant with an initial compressive modulus that decreases 3-6 months after implantation.

In one embodiment, the compressive modulus of the implant is 0.1 kPa to 10 MPa at 5 to 15% strain, more preferably 1 MPa to 10 MPa at 5 to 15% strain, and even more preferably 1 MPa to 5 MPa at 5 to 15% strain.

In embodiments, the planes of filaments present in the macroporous network of the nipple implant are formed from a polymeric composition. The polymeric composition preferably has one or more of the following properties: (i) an elongation at break greater than 100%; (ii) an elongation at break greater than 200%; (iii) a melting temperature of 60° C. or higher, (iv) a melting temperature higher than 100° C., (v) a glass transition temperature of less than 0° C., (vi) a glass transition temperature between −55° C. and 0° C., (vii) a tensile modulus less than 300 MPa, and (viii) a tensile strength higher than 25 MPa.

In embodiments, the planes of filaments present in the macroporous network of the nipple implant have one or more of the following properties: (i) breaking load of 0.1 to 200 N, 1 to 100 N, or 2 to 50 N; (ii) elongation at break of 10% to 1,000%, more preferably 25% to 500%, and even more preferably greater than 100% or 200%, and (iii) elastic modulus of 0.05 to 1,000 MPa, and more preferably 0.1 to 200 MPa.

In order to allow tissue ingrowth into the macroporous network of the implant, the macroporous network should have a strength retention long enough to permit cells to invade the implant's macroporous network and proliferate. In embodiments, the macroporous network of the implant has a strength retention of at least 25% at 2 weeks, more preferably at least 50% at 2 weeks, and even more preferably at least 50% at 4 weeks. In other embodiments, the macroporous network of the implant is designed to support mechanical forces acting on the implant, and to allow a steady transition of mechanical forces from the macroporous network to regenerated host tissues. In particular, the macroporous network of the implant is designed to support mechanical forces acting on the implant, and to allow a steady transition of mechanical forces to new host tissues.

### D. Other Features of the Implants

The implants or macroporous networks of the implants may be trimmed or cut with scissors, blades, other sharp cutting instruments, or thermal knives in order to provide the desired implant or macroporous network shapes. The implants or macroporous networks can also be cut into the desired shapes using laser-cutting techniques. This can be particularly advantageous in shaping filament-based implants because the technique is versatile, and importantly can provide shaped implants and macroporous networks without sharp edges.

The implants may comprise retainers, such as barbs or tacks, so that the implant can be anchored in the body without the use of sutures. The implants preferably contain the retainers on the circumference of the first circular base of the implant or on the flange. In embodiments, the retainers are preferably located on the implant to allow the implant to be anchored to the breast.

The implant may comprise suture tabs so that the implants can be anchored in the body using for example sutures and or staples. The number of tabs may vary. In embodiments, the implant comprises 1, 2, 3, 4, tabs or more. The tabs attached to the implant must have sufficient strength retention in vivo to resist mechanical loads, and to allow sufficient ingrowth of tissue into the implant in order to prevent subsequent movement of the implant after implantation. In a preferred embodiment, the suture pullout strength of the tabs attached to the implant, is greater than 10 N, and more preferably greater than 20 N.

### E. Implant Coatings and Fillings

The macroporous network of the implant comprises a network wherein there is a continuous path through the network which encourages and allows tissue ingrowth into the implant. The continuous path also allows the entire macroporous network to be coated with one or more of the following: bioactive agents, collagen, hyaluronic acid or derivative thereof, additives, and cells, including fat and fat cells.

Macroporous networks with low infill densities, for example, less than 60%, or 5-25%, are preferred because they provide a large void space that can be occupied, for example, by cells, collagen, and bioactive agents, including fat, lipoaspirate, adipose cells, fibroblast cells, and stem cells. In one embodiment, 25% to 100% and more preferably 75% to 100% of the void space of the implant's macroporous network is filled with one or more of the following: cells, collagen, and bioactive agents, including fat, lipoaspirate, adipose cells, fibroblast cells, and stem cells.

The cells and other compositions, such as collagen, hyaluronic acid or derivative thereof, and other bioactive agents, may be coated on the macroporous network prior to implantation, after implantation, or both before and after implantation.

In embodiments, the implants are fabricated with coatings and or some or all of the macroporous network is used as a carrier. For example, the macroporous network may be fabricated by populating some or all of the void space of the macroporous network with one or more of the following: cells, including autograft, allograft and xenograft cells. Examples of cells that can be inserted into the void spaces of the implant's macroporous network, and coated on the surfaces of the macroporous network, include fibroblast cells, and stem cells. In a preferred embodiment, autologous fat, fat lipoaspirate, or injectable fat, is coated on the implant's macroporous network and or inserted into void space of the implant's macroporous network. In yet another embodiment, the implant's macroporous network can be coated or partially or fully filled with one or more bioactive agents. Particularly preferred bioactive agents that can be coated on the implant's macroporous network or used to partially or completely fill the implant's macroporous network include collagen and hyaluronic acid or derivative thereof. In other embodiments, the implant's macroporous network may be coated with one or more antibiotics.

Any suitable method can be used to coat the implant's macroporous network and fill its void space with cells, bioactive agents and other additives. In embodiments, the implant's macroporous network is filled or coated with cells, bioactive agents and other additives by injection, spraying, or dip-coating. Collagen may be applied to the implant's macroporous network by coating and freeze-drying. In a particularly preferred embodiment, the implant's macroporous network may be coated or partially or completely filled with cells, bioactive agents and or other additives by injection using needles that can be inserted into the macroporous network of the implant preferably without damaging the macroporous network. In one embodiment, the needles used for injection of cells, fat, fat lipoaspirate, bioactive agents, collagen, hyaluronic acid or derivative thereof, and other additives have outer diameters between 0.5 mm and 5 mm.

### IV. Methods for Implanting the Implants

In embodiments, the implant is implanted into the body. Preferably, the implant is implanted into a site of reconstruction, remodeling, repair, and or regeneration. In embodiments, the implant is implanted in a patient to form a nipple, reshape a nipple, reconstruct a nipple, modify a nipple, or replace tissue that has been damaged or surgically removed.

In a preferred embodiment, the implant is implanted into a tissue enclosure on the breast mound of a patient. In embodiments, connective tissue and or vasculature will invade the macroporous network of the implant after implantation. In a particularly preferred embodiment, the implant comprises absorbable materials, and connective tissue and or vasculature will also invade the spaces where the absorbable materials have degraded. The pores of the macroporous network may be colonized by cells prior to implantation or, more preferably, following implantation, and the pores of the implant's macroporous network invaded by tissue, blood vessels or a combination thereof.

The implant's macroporous network may be coated or filled with transplantation cells, stem cells, fibroblast cells, adipose cells, and or tissues prior to implantation, or after implantation. In embodiments, the implant's macroporous network is coated or filled with differentiated cells prior to, or subsequent to, implantation. Differentiated cells have a specific form and function. An example is a fat cell. In embodiments, the implant's macroporous network is populated with cells by injection, before or after implantation, and more preferably by using needles that do not damage the macroporous network of the implant. The implant's macroporous network may also be coated or filled with platelets, extracellular adipose matrix proteins, gels, hydrogels, and bioactive agents prior to implantation. In an embodiment, the implant's macroporous network may be coated with antibiotic prior to implantation, for example, by dipping the implant in a solution of antibiotic.

The implants may be used to deliver autologous cells and tissue to the patient. The autologous tissue is preferably one or more of the following: autologous fat, fat lipoaspirate, injectable fat, adipose cells, fibroblast cells, and stem cells.

The implants may be used to deliver fat tissue to a patient. In a particularly preferred embodiment, autologous fatty tissue is prepared prior to, or following, implantation of the implant, and is injected or otherwise inserted into or coated on the implant's macroporous network prior to or following implantation of the implant. The autologous fatty tissue is preferably prepared by liposuction at a donor site on the patient's body. After centrifugation, the lipid phase containing adipocytes is then separated from blood elements, and combined with the implant's macroporous network prior to implantation, or injected, or otherwise inserted into the implant's macroporous network following implantation. In an embodiment, the implant's macroporous network is injected with, or filled with, a volume of lipoaspirate that represents 1% to 50% of the total volume of the macroporous network, and more preferably 1% to 20% of the total volume of the macroporous network.

In another embodiment, lipoaspirate fatty tissue taken from the patient may be mixed with a biological or synthetic matrix, such as very small fibers or particles, prior to adding the lipoaspirate to the implant's macroporous network. In this embodiment, the added matrix serves to hold or bind micro-globules of fat, and disperse and retain them within the macroporous network of the implant.

In an embodiment, an implant is implanted on the tissue mound of a breast. In an embodiment, implants are implanted on the tissue mounds of both breasts of a patient.

In a particularly preferred embodiment, the implant is implanted in a patient that has undergone a mastectomy.

In an embodiment, the implant is inserted in a tissue enclosure formed at the site of nipple reconstruction.

In a preferred embodiment, the implant is implanted by a method comprising: making an incision in a patient to create a tissue enclosure that is configured to receive a nipple implant; and inserting the nipple implant into the tissue enclosure, wherein the tissue enclosure is configured to conform around the nipple implant. In embodiments, the method of implanting the implant comprises configuring an incision to create tissue flaps with opposable edges, such that when the edges are brought together the tissue flaps form a void for receiving the nipple implant so that the inner surface of the tissue flaps are in contact with the nipple implant. In embodiments, the method of implanting the implant comprises making an incision with a CV-flap incision path, a S-flap incision path, or a star-flap incision path.

In an embodiment, the implant is implanted by a method comprising: (i) making one or more incisions on the breast mound of a reconstructed patient breast to create free-moving skin flaps, (ii) manipulating and fixating the skin flaps to produce a projecting tissue enclosure, (iii) inserting a nipple implant into the tissue enclosure, (iv) opposing the patient's tissue against the external surface of the nipple implant, and (v) fixating the tissue enclosure to enclose the implant within the tissue enclosure. In an embodiment, the method further comprises suturing the skin flaps to form a projecting tissue enclosure. In an embodiment, the method further comprises suturing the tissue enclosure to enclose the implant within the tissue enclosure. In embodiments, the tissue enclosure is sized so that there is little if any dead space between the implant and patient tissue. In embodiments, the tissue enclosure is sized to conform to the volume of the implant.

In embodiments, the method of implantation comprises implanting the first end of the cylindrical shape of the implant posterior to the second end of the cylindrical shape of the implant. In a particularly preferred embodiment, the method of implantation comprises implanting the first end of the cylindrical shape of the implant posterior to the second end of the of the implant. In embodiments, the hemispherical shape of the implant is implanted beneath the skin of the patient, and the first end of the cylindrical shape of the implant is implanted on the breast mound of the patient.

In embodiments, the implant comprises a flange on the first end of the cylindrical shape of the implant, and the method of implantation comprises implanting the flange of the implant on the breast mound and posterior to the second end of the cylindrical shape of the implant.

The implant's macroporous network may be coated or filled with cells and tissues prior to, or subsequent to, implantation, as well as with cytokines, platelets and extracellular adipose matrix proteins. The implant's macroporous network may also be coated or filled with other tissue cells, such as stem cells genetically altered to contain genes for treatment of patient illnesses.

In an embodiment, the implant has properties that allows it to be delivered by minimally invasive means through a small incision. The implant may, for example, be designed so that it can be rolled, folded or compressed to allow delivery through a small incision. In an embodiment, the implant has a three-dimensional shape and shape memory properties that allow it to assume its original three-dimensional shape unaided after it has been delivered through an incision and into a tissue enclosure. For example, the implant may be temporarily deformed by rolling it up into a small diameter cylindrical shape, delivered using an inserter, and then allowed to resume its original three-dimensional shape unaided in vivo.

### EXAMPLES

Embodiments of the present invention will be further understood by reference to the following non-limiting examples.

### Example 1: 3D Printed Nipple Implant with an Internal Filament Structure Made from Printed P4HB Filaments, No Flange, and a Shell with 100% Infill and Circular Pores

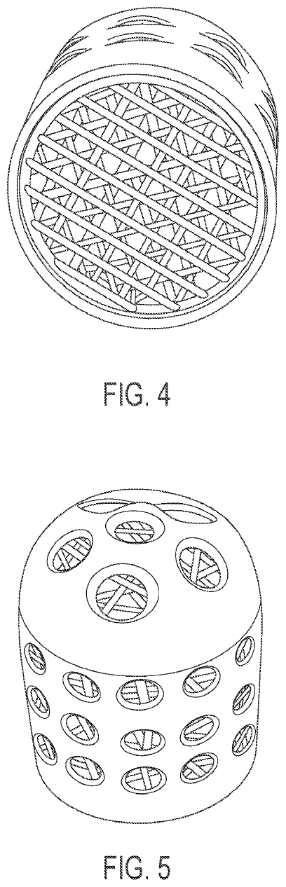

Pellets of poly-4-hydroxybutyrate, P4HB, (Tepha, Inc., Mw 300 kDa) were loaded into the hopper of a melt extrusion deposition (MED) based 3D printer comprising a horizontal extruder feeding into a vertical extruder fitted with a vertical plunger, and a movable stage. The movable stage and the printer head were enclosed inside a build chamber. The pellets had an average diameter of 3.5 mm, moisture content of less than 100 ppm, and were kept dry in the hopper using a purge of air dried through a silica bed. The temperature profile of the horizontal extruder was set to 10° C. in the build chamber; 100° C. in the first barrel zone, 135° C. in second barrel zone; and 185° C. in the extrusion zone (printhead temperature). The back pressure was set to 50 bars (5 MPa) and the melt screw speed was set to 4 m/min. The recovery stroke was 6 mm, deco speed was 2 mm/sec, Deco stroke was 4 mm, and a drop aspect ratio of 1.13. The diameter of the nozzle orifice of the extruder (printhead) was 0.2 mm. The 3D printer was loaded with an STL file to print the open porous scaffold structure of the implant shown in FIGS. 4 and 5. The printed nipple implant height was 12 mm and the base cylindrical diameter was 11.2 mm. The implant shell was printed at 50 drops/sec and 20% infill. The shell was 0.7 mm thick with 100% infill and had circular pores of 3 mm diameter spread at an equal distance of 4 mm apart. The inside of the implant was printed at 240 drops/sec and 10% infill density.

### Example 2: 3D Printed Nipple Implant with an Internal Filament Structure Made from Printed P4HB Filaments and Porous Flange

A 3D printed nipple implant was prepared as described in Example 1, except a porous flange was printed using the same print settings with an outer diameter of 13.2 mm, a height of 3 mm, 100% infill density, and 3 mm macropores.

### Example 3: 3D Printed Nipple Implant with an Internal Filament Structure Made from Printed P4HB Filaments, No Flange, and a Shell with 60% Infill

A 3D printed nipple implant was prepared as described in Example 1 with the same structure shown in FIG. 4, except for the shell had an infill of 60% instead of 100% and the shell did not have circular pores.

### Example 4: 3D Printed Nipple Implant with an Internal Filament Structure Made from Printed Poly(Butylene Succinate) PBS Filaments, No Flange, and a Shell with 100% Infill

A 3D printed nipple implant was prepared with the same structure as described in Example 1 except polybutylene succinate (PBS) pellets were used instead of P4HB pellets. The same 3D printing equipment and STL file were used but with the following print settings: the temperature profile of the horizontal extruder was set to 80° C. in the build chamber; 110° C. in the first barrel zone, 150° C. in second barrel zone; and 200° C. in the extrusion zone (printhead temperature). The back pressure was set to 50 bars (5 MPa) and melt extrusion speed was 4 m/min. The recovery stroke was 6 mm, deco speed was 2 mm/sec, Deco stroke was 4 mm, and a drop aspect ratio of 1.64.

### Example 5: 3D Printed Nipple Implants with an Internal Filament Structure Made from Printed P4HB Filaments with 20, 25 and 30% Infill, a Porous Flange, and No Outer Shell

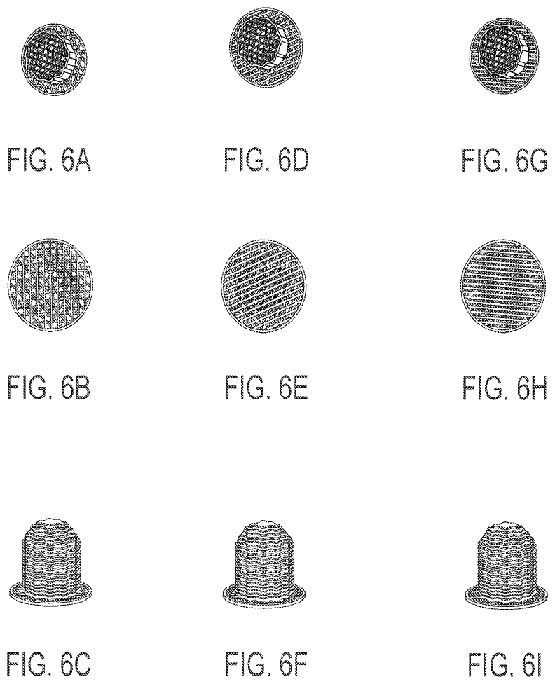

Pellets of poly-4-hydroxybutyrate, P4HB, (Tepha, Inc., Mw 300 kDa) were loaded into the hopper of a melt extrusion deposition (MED) based 3D printer comprising a horizontal extruder feeding into a vertical extruder fitted with a vertical plunger, and a movable stage. The movable stage and the printer head were enclosed inside a build chamber. The pellets had an average diameter of 3.5 mm, moisture content of less than 100 ppm, and were kept dry in the hopper using a purge of air dried through a silica bed. The temperature profile of the horizontal extruder was set to 12-14° C. in the build chamber; 100° C. in the first barrel zone, 135° C. in second barrel zone; and 185° C. in the extrusion zone (printhead temperature). The back pressure was set to 50 bars (5 MPa) and the melt screw speed was set to 4 m/min. The recovery stroke was 6 mm, deco speed was 2 mm/sec, Deco stroke was 4 mm, and a drop aspect ratio of 1.13. The diameter of the nozzle orifice of the extruder (printhead) was 0.2 mm. The 3D printer was loaded with STL files to print the three open porous scaffold structures of the implants shown in FIG. 6A-I. The printed nipple implant heights were 12 mm and the base cylindrical diameters were 10.2 mm. Each implant was printed with a porous flange. Each implant was printed with layers of parallel filaments with each layer of filaments angled at forty-five degrees (45°) from an adjacent layer of filaments. The average diameter of the printed filaments was 200 microns. The infill was printed at 240 drops/sec with each implant having a different amount of infill: 20% infill shown in FIG. 6A (top view), FIG. 6B (bottom view) and FIG. 6C (side view), 25% infill shown in FIG. 6D (top view), FIG. 6E (bottom view) and FIG. 6F (side view), and 30% infill shown in FIG. 6G (top view), FIG. 6H (bottom view) and FIG. 6I (side view). The mechanical properties of each implant when subject to cyclic compression in the radial direction (x-direction) are reported in Table 3. As the infill of the implant was increased from 20 to 30%, the compressive load at 17% strain increased from 22.407 N to 53.275 N, the stiffness at 4-10% strain increased from 1.19 to 2.009 N/mm, and the stiffness at 14-17% strain increased from 1.762 to 5.867 N/mm. The surface area per volume of the implants were determined for each implant. The surface area per volume of the implants with 20%, 25% and 30% infill were 3.2, 3.9 and 4.3 mm2/mm3, respectively.

TABLE 3
Cyclic compression in the radial direction of P4HB 3D Printed Nipple
Implants with Flanges, No Outer Shells, and Varying Infill

| | Avg Max Compressive | Stiffness @ 4-10% | Stiffness @ 14-17% |
|---|---|---|---|
| Sample | Load @ 17% Strain (N) | Strain, (N/mm) | Strain, (N/mm) |
| 20% Infill | 22.407 | 1.190 | 1.762 |
| 25% Infill | 40.992 | 1.596 | 3.677 |
| 30% Infill | 53.275 | 2.009 | 5.867 |

1. A 3D printed nipple implant comprising a macroporous network with an open cell structure comprising a cylindrical shape for placement under the skin of the patient, wherein the cylindrical shape has first and second ends each with circular bases, a height measured between the two circular bases, a circumference, and wherein the macroporous network comprises at least two adjacent parallel planes of filaments bonded to each other.

2. The implant of claim 1, wherein the implant further comprises a hemispherical shape at the second end of the cylindrical shape, and optionally, having a stiffness less than or equal to 20 kPa.

3. The implant of claim 1, wherein the macroporous network is enclosed by a shell or coating.

4. The implant of claim 1, wherein the implant further comprises a flange component protruding from the circular base on the first end of the cylindrical shape.

5. The implant of claim 1, wherein the filaments are arranged as chords with endpoints.

6. The implant of claim 1, wherein the filaments are not continuous.

7. The implant of claim 5, wherein the endpoints of the filaments in a plane of filaments are not connected to another filament that is in the same plane of filaments.

8. The implant of claim 1, wherein the filaments have endpoints on the circumference of the cylindrical shape, and do not form arcs on the circumference of the cylindrical shape.

9. The implant of claim 1, wherein the macroporous network is at least partly filled with a hydrogel.

10. The implant of claim 1, wherein the macroporous network comprises an absorbable polymer.

11. The implant of claim 1, wherein at least two parallel planes of filaments have the same orientation in adjacent planes or nonadjacent planes.

12. The implant of claim 1, wherein a first parallel plane of filaments is organized in a first geometrical orientation, and a second parallel plane of filaments is arranged in a second geometrical orientation such that the implant comprises a macroporous network of crisscrossed filaments.

13. The implant of claim 12, wherein the implant further comprises a third parallel plane of filaments, and the filaments in the first, second and third parallel planes form pores with a triangular shape.

14. The implant of claim 1, wherein the angle between the filaments in the parallel planes is selected from one of the following: between 1 and 90 degrees, or 18, 20, 30, 36, 45 or 60 degrees.

15. The implant of claim 1, wherein the macroporous network comprises a plurality of macropores, and the macropores have an average diameter or average width of 75 to 2,000 microns.

16. The implant of claim 1, wherein the filaments have one or more of the following properties: an average diameter or average width of 10 μm to 5 mm, a breaking load of 0.1 to 200 N, an elongation at break of 10 to 1,000%, and an elastic modulus of 0.05 to 1,000 MPa.

17. The implant of claim 10, wherein the absorbable polymer has one or more of the following properties: (i) an elongation at break greater than 100%; (ii) an elongation at break greater than 200%; (iii) a melting temperature of 60° C. or higher, (iv) a melting temperature higher than 100° C., (v) a glass transition temperature of less than 0° C., (vi) a glass transition temperature between −55° C. and 0° C., (vii) a tensile modulus less than 300 MPa, and (viii) a tensile strength higher than 25 MPa.

18. The implant of claim 1, wherein the macroporous network has an infill density of filaments of between 1% and 60%, or between 5% and 25%.

19. The implant of claim 3, wherein the shell comprises a stack of concentric filaments.

20. The implant of claim 1, wherein the implant further comprises one or more of the following: autologous fat, fat lipoaspirate, injectable fat, adipose cells, fibroblast cells, stem cells, gels, hydrogels, hyaluronic acid, collagen, antimicrobial agent, antibiotic agent, and bioactive agent.

21. The implant of claim 10, wherein the absorbable polymer comprises, or is prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 4-hydroxybutyric acid, 4-hydroxybutyrate, 3-hydroxyoctanoate, E-caprolactone, 1,4-butanediol, 1,3-propane diol, ethylene glycol, glutaric acid, malic acid, malonic acid, oxalic acid, succinic aid, or adipic acid, or the absorbable polymer comprises poly-4-hydroxybutyrate or copolymer thereof, or poly(butylene succinate) or copolymer thereof.

22. The implant of claim 1, wherein the implant is absorbable.

23. The implant of claim 1, wherein the implant is manufactured by a process selected from the group comprising: (i) forming the macroporous network by 3D printing the parallel planes of filaments, (ii) forming the macroporous network by melt extrusion deposition 3D printing, and (iii) bonding the filaments in adjacent parallel planes by 3D printing.

24. The implant of claim 1, wherein the implant has a compressive modulus of 0.1 kPa to 10 MPa at 5 to 15% strain.

25. The implant of claim 1, wherein the base of the first end comprises an open bottom aperture.

26. A method of manufacturing a nipple implant comprising a load bearing macroporous network with an open cell structure comprising a cylindrical shape for placement under the skin of the patient, wherein the cylindrical shape has first and second ends each with circular bases, a height measured between the two circular bases, a circumference, wherein the macroporous network comprises at least two adjacent parallel planes of filaments bonded to each other, and wherein the method comprises forming the macroporous network by one of the following: (i) forming at least two parallel planes of filaments from a polymeric composition by 3D printing of the filaments, and (ii) forming at least two parallel planes of filaments from a polymeric composition by melt extrusion deposition 3D printing.

27-38. (canceled)
